(19) United States
(12) Patent Application Publication
(10) Pub. No.: US 2018/0075208 A1
(43) Pub. Date: Mar. 15, 2018

(54) Systems and Methods For Placing A Participant In A Disease Prevention, Monitoring Program Milestones, and Third Party Reporting and Payment

(71) Applicant: Solera Health, Inc., Phoenix, AZ (US)

(72) Inventors: Brenda Schmidt, Phoenix, AZ (US); James Prevallet, Phoenix, AZ (US)

(21) Appl. No.: 15/783,907

(22) Filed: Oct. 13, 2017

Related U.S. Application Data
(63) Continuation-in-part of application No. 14/808,956, filed on Jul. 24, 2015.
Application No. 14/808,956 is a continuation-in-part of application No. 15/049,723, filed on Feb. 22, 2016.

(51) Int. Cl. G06F 19/00

(52) U.S. Cl. CPC G06F 19/3431; A61B 5/7275; G06F 19/3437; G06F 19/3443

(57) ABSTRACT

Various embodiments provide a computer-implemented data architecture for a three-sided marketplace consisting of a consumer, a disease prevention program provider, and a health insurance plan provider

### CROSS-RELATED APPLICATIONS

This patent application is a continuation-in-part of U.S. patent application Ser. No. 14/808,956, filed Jul. 24, 2015 and published on Jan. 26, 2017 as US Patent Application Publication No. 2017/0024544, which is incorporated by reference herein.

This patent application is a continuation-in-part of U.S. patent application Ser. No. 15/049,723 filed Feb. 22, 2016 and published on Jan. 26, 2017 as US Patent Application Publication No. 2017/0024546, which is incorporated by reference herein.

### TECHNICAL FIELD

The present invention relates, generally, to a data architecture for providing a personalized precision prevention program for a consumer and, more particularly, to systems and methods for selecting a disease prevention program (“DPP”) and an optimal DPP provider based on a consumer risk assessment and individual consumer preferences and enrolling the consumer in the DPP with the optimal DPP provider.

### BACKGROUND

The evolving U.S. health care system presents opportunities for improving population health. By reversing the escalating epidemic of chronic diseases such as obesity, diabetes, and cardiovascular disease, population health offers better care for individual patients, improved aggregate health for the population, and lower overall healthcare costs. A key component of population health involves linking clinical care with community-based prevention programs and related social services. Traditional medical education, research, and practice have focused on the care of the individual. Shifting the emphasis to embracing population-based principles can have a greater effect on long term health and wellness, particularly in the prevention of chronic disease.

At present, healthcare providers supply their eligible patients with a list of non-medical organizations offering a disease prevention program (“DPP”), relying on the patient to follow up directly with a community based organization (“CBO”). Unfortunately, this this type of “opt-in” approach tends to result in significantly lower enrollment, in part because prevention programs offered by CBOs are typically not covered by health insurance plans. More recently, some health insurance plans have begun to include certain DPPs as covered benefits for their insured members. However, most DPP providers lack the infrastructure to submit medical claims for their services, and it would be cumbersome and costly for health plans to independently contract with each DPP, whether it is a community-based non-medical organization or a virtual (e.g., on-line) DPP provider.

It is also known that patient behavior is a key metric in the success of chronic disease prevention programs. Consequently, disease prevention programs should strive to deliver against patients' needs, preferences and expectations. “One size fits all” programs and delivery methods have had limited success because all patients are not alike—even when they share a common health condition.

Presently known chronic disease prevention program delivery models lack sufficient understanding of consumer preferences and how to effectively influence their choices.

Systems and methods are thus needed which overcome these limitations. Various desirable features and characteristics will also become apparent from the subsequent detailed description and the appended claims, taken in conjunction with the accompanying drawings and this background section.

### BRIEF SUMMARY

The present invention provides a computer implemented, integrated transaction architecture for placing a participant in a disease prevention program (“DPP”), monitoring program milestones, and remitting milestone payments to the DPP provider. Various embodiments include reporting the completed milestones to a third party payer, and receiving payments from the payer for the completed milestones.

Some embodiments employ a three-sided marketplace metaphor, in which an integrator facilitates a series of transactions for ordering, fulfilling, and paying for disease prevention programs. In this model the consumers, program providers, and payers interface with the integrator at different times from enrollment through completion of and payment for the DPP.

Various embodiments provide systems and methods for selecting a DPP and an optimal DPP provider based on a consumer risk assessment and individual consumer preferences, including the application of predictive analytics to determine the best-fit DPP and the optimal DPP provider.

Exemplary integrator systems employ a series of algorithms and/or branched logic to determine one or more “best match” programs for each program participant, allowing the consumer to explore options based on his or her expressed preferences. Successful application of these insights can positively drive program engagement and influence health and wellness behaviors, and support ongoing retention and successful program completion.

In an embodiment, the integrator system includes a database which stores a diverse network of program providers, each delivering similar evidence-based programs having variations in how the programs are delivered. Participant preferences (which may be programmatically weighted) are applied to the database. Using predictive analytics, the integrator system determines the profile of the “ideal participant” for each program provider based on the foregoing variables and individual participant characteristics. This profile represents a hypothetical participant most likely to be successful in each program based on delivery methodology.

After matching participant-specific data to various ideal participant profiles, the integrator system algorithmically selects the DPP provider best suited to the consumer. In this context, the consumer personal profile data may include, for example, patient contact information (including zip code), demographics, socio-economic factors, psychographics, health information, health care utilization, claims data, electronic medical record data, prescription history, and purchasing data.

The integrator system may also be configured to track meaningful engagement between the participant and the assigned DPP. For example, meaningful engagement contemplates the satisfaction of predetermined milestones associated with the DPP, which can then be used to trigger a milestone payment from the payer to the DPP program provider (e.g., a CBO). Accordingly, meaningful engagement is an event, which initiates an action. For example, upon meaningful engagement, the DPP provider is sent a payment. For example, upon meaningful engagement, a claim is sent to the payer. Meaningful engagement metrics can be tracked for a plurality of participants and a plurality of CBOs to improve the system.

Various other embodiments, aspects and features are described in greater detail below.

### BRIEF DESCRIPTION OF THE DRAWING FIGURES

The present disclosure will become more fully understood from the description and the accompanying drawings, wherein:

FIG. 1 is a schematic block diagram of an exemplary integrator-centric system for facilitating the provision of disease prevention programs in accordance with various embodiments;

FIG. 2 is a schematic block diagram of an exemplary system for determining a best-fit DPP and corresponding optimal DPP provider for a consumer in accordance with various embodiments;

FIG. 3 is a process flow diagram illustrating an exemplary use case involving a consumer, a payer, a DPP provider, and an integrator in accordance with various embodiments;

FIG. 4 is a schematic block diagram of an exemplary system for identifying a plurality best-fit DPPs, each for a different disease, and a plurality of optimal DPP providers, for each disease, and assigning the DPP and provider to a consumer in accordance with various embodiments;

FIG. 5 is a schematic block diagram of an integrator system including an integrator computer module having a processor, a database of DPP providers, and a database of participants received from a plurality of sources in accordance with various embodiments;

FIG. 6 is a schematic block diagram of an exemplary integrator system including an integrator application engine configured to exchange data with a customer relationship management (“CRM”) module in accordance with various embodiments;

FIG. 7 is a schematic block diagram detailing various functional modules associated with and the operation of an exemplary integrator application engine in accordance with various embodiments;

FIG. 8 is an exemplary spread sheet of providers within a managed provider network in accordance with various embodiments;

FIG. 9 is a schematic block diagram illustrating a plurality of DPP providers, each having an associated ideal profile in accordance with various embodiments;

FIG. 10 is a flow diagram illustrating an exemplary process for creating ideal participant profiles for a plurality of DPP providers in accordance with various embodiments;

FIG. 11 is a schematic block diagram illustrating a process by which a heterogeneous population is segmented into smaller homogeneous sub-groups characterized by segment-specific variables in accordance with various embodiments;

FIG. 12 is a flow diagram illustrating a process by which a best fit program provider is determined for a candidate participant in accordance with various embodiments; and

FIG. 13 is a flow diagram illustrating a process by which an integrator system matches a participant to a DPP and monitors program engagement and outcome in accordance with various embodiments.

The drawings described herein are for illustrative purposes only of selected embodiments and not all possible implementations, and are not intended to limit the scope of any embodiment disclosed herein or any equivalents thereof.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

Various embodiments of the present invention relate to systems and methods for linking a consumer (also referred to herein as a patient, a participant, candidate, or a user) with (typically non-medical) community-based organizations (“CBOs”) or other virtual providers to provide disease prevention programs (“DPPs”) based on the consumer's input into a risk assessment and into a survey of program preferences. The present invention further contemplates systems and methods which facilitate determining a best-fit DPP for a particular consumer based on the consumer's input.

In various embodiments, an integrator manages a network of DPP providers, which can include CBOs that have been vetted, validated, and approved by the integrator or an accrediting agency such as the government. The integrator also processes a candidate consumer, who is eligible for one or more DPPs, where the DPPs are typically paid for by a health plan as a “covered” preventative benefit under a statutory scheme such as the Affordable Care Act (ACA), or pursuant to related policy objectives. In accordance with various embodiments, it is the integrator—as opposed to the DPP provider—which has a contractual relationship with the health plan, health system or healthcare provider (collectively referred to herein as the “payer”) to secure payment from the payer. The integrator remits payment the DPP provider upon satisfaction of program milestones by the consumer enrolled in the DPP. Consequently, a consumer and the CBO are incented to cooperate with the integrator for selection of a DPP provider and enrollment with the DPP provider, as opposed to the consumer directly engaging an unknown DPP provider.

In an embodiment, a payer typically reimburses a DPP provider, via the integrator, based on a pay-for-performance model using a series of events, such as, for example, predetermined milestones for meaningful engagement by the consumer and/or desired outcomes of the DPP. Accordingly, an integrator computer system can be generally configured as a finite state machine, which is driven by the series of events to perform an action such as, for example, enrolling a consumer, preparing and sending claims to the payer, receiving payments from the payer, collecting data, reporting milestones, and sending payments to the CBO.

The present invention further identifies best fit programs from a wide variety of treatment providers for consumers already identified as candidates for treatment.

Some embodiments provide methods and systems for determining an optimum DPP and/or DPP provider, for which the consumer is most likely to successfully complete, from among many with essentially the same content, based on matching a consumer's success metrics with ideal participant profiles associated with various DPP providers and programs.

Such systems can use predictive analytics and machine learning to pair a particular candidate with a specific one of several programs having analogous content, based on a prediction that the candidate will do well in the optimally selected program.

Some embodiments provide systems and methods for providing a personalized precision prevention program for a consumer, which may be event driven by program milestones that mark the progress of the consumer through the program and initiate corresponding payments to the program provider. The personalize precision prevention program can be designed to enhance and encourage the consumer to have meaningful engagement with the program, which increases the probability the consumer completes a DPP.

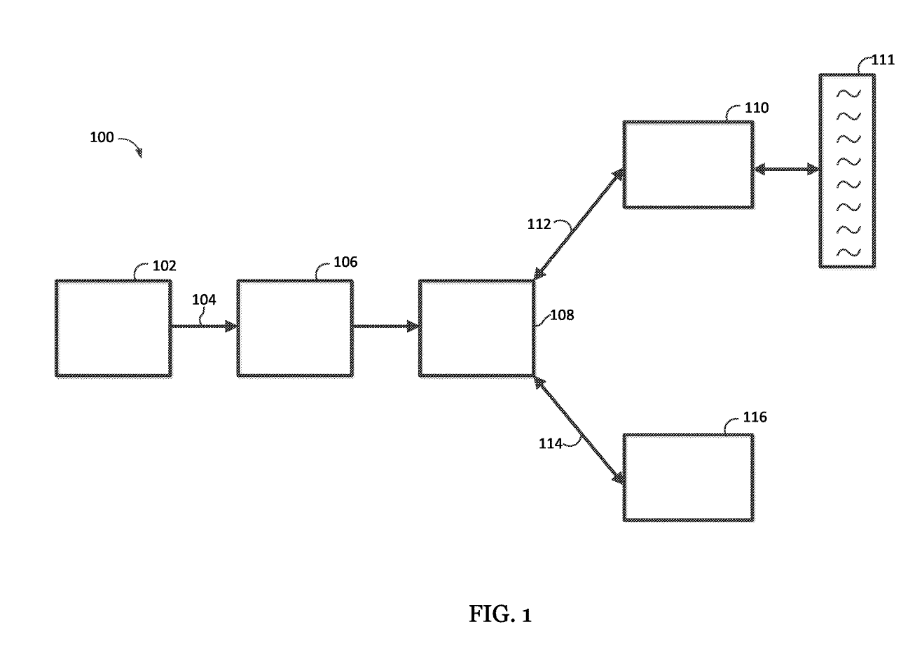

More particularly and referring now to FIG. 1, an exemplary integrator-centric system 100 for delivering at least one disease prevention program (“DPP”) includes a clinical provider 102 configured to refer 104 a consumer 106 to an integrator 108. The clinical provider 102 includes but is not limited to a doctor or a hospital. The refer 104 can be in the form of a professional referral or a prescription. The consumer 106 can include a patient, a client of a medical service, a program participant, a candidate, and/or a user. However, the consumer 106 can act as an agent for a third party whereby the consumer 106 can enroll the third party into a DPP 110.

An early step in the process of matching a consumer 106 to a best-fit DPP no involves determining whether the consumer 106 is eligible for one or more DPPs 110 based on objective criteria, as defined by the consumer's health plan (e.g., payer 116).

The integrator 108 accesses a database in of DPP providers and recommends a “best fit” DPP 110 based on, for example, correlation between a personal profile entered by the consumer 106 and one of a plurality of ideal participant profiles, each associated with a DPP provider. The integrator 108 enrolls the consumer 106 into the best-fit DPP 110. As described in greater detail below, the integrator 108 monitors 112 the consumer's compliance with the DPP 110, and processes a claim for payment 114 from a payer 116. The payer 116 can be an insurance company, Medicaid, Medicare, a health system, or health plan administrator. The integrator 108 may be configured to process a claim for payment by performing one of more of the steps of: submitting a claim to the payer 114, receiving approval for the claim from the payer 116, invoicing the payer 116, and receiving the payment for the claim from the payer 116. The integrator 108 can send the initial claim to the payer 116 upon enrollment of the consumer 106 in the DPP 110.

In accordance with various embodiments, the system 100 programmatically determines an ideal personality profile based on the outcomes of the most successful participants for each DPP provider. Each DPP provider can have one or more vehicles for delivering a DPP 110. For example, a DPP provider can offer a qualifying DPP 110 in a group session and alternatively, can offer another qualifying DPP 110 using a virtual interface.

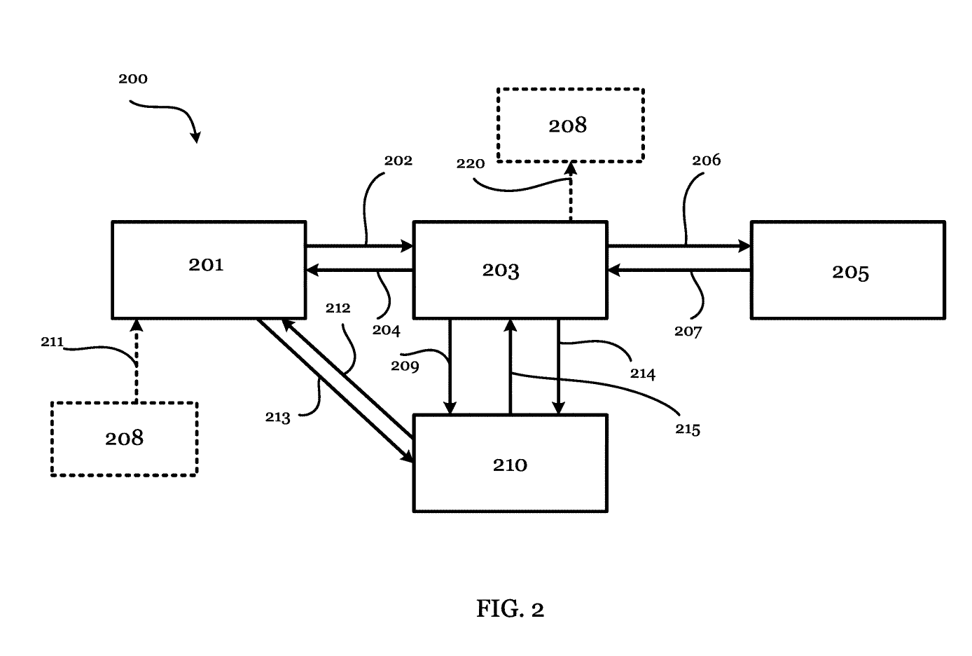

With reference to FIG. 2, an exemplary system 200 for implementing the aforementioned three sided marketplace metaphor includes an integrator 203, a consumer 201, a payer 205, and a DPP provider (CBO) 210. In this regard, each of the consumer 201, the DPP provider 210, and the payer 205 interface with the integrator 203 at different events from enrolling the consumer in an appropriate DPP, and thereafter through program monitoring, completion, and payment.

The consumer 201 initiates a sequence of events (collectively referred to herein as the transaction) by making contact with the integrator 203 for enrollment in a DPP. The consumer 201 inputs data 202 responsive to a health risk assessment and a personal preference survey. These data are analyzed by the integrator's computer system, for example by using algorithms to determine if the consumer 201 is eligible for a DPP. Data 202 may be in the form of Yes or No answers and can be computed by employing a finite state machine or a modification thereof. If the consumer 201 is eligible for the DPP, then the integrator's computer system determines the best-fit DPP provider 210 using analytics to compare the personal preferences of the consumer 201 to ideal personal preferences for each of a plurality of DPP providers. In addition, the consumer 201 can enter a location that the consumer 201 will be before traveling to the DPP provider 210, for example, home, work, school, the gym, or the office. The location of the consumer 201 is compared to a location of each of the plurality of DPP providers and then analyzed. Based on this analysis of personal preferences and locations, a best-fit DPP provider 210 is identified. The personal preferences can be used to determine a best-fit DPP 210. The integrator 203 enrolls the consumer 201 in the best-fit DPP with the best-fit DPP provider 210. Notice of enrollment 204 is sent to the consumer 201. A second notice of enrollment 209 is sent to the best-fit DPP provider 210.

Upon enrollment, the integrator 203 sends a claim 206 to payer 205. The payer 205 sends an approval or payment 207 to integrator 203. The consumer 201 participates 213 in the DPP and the DPP provider 210 delivers the DPP content 212 to the consumer 201. The DPP provider 210 reports 214 progress and other data to the integrator 203. Once the integrator 203 identifies that the consumer 201 has satisfied a milestone or completed the DPP, the integrator 203 sends a payment 215 to the DPP provider 210, which ends the transaction or advances the transaction to the next milestone.

Some embodiments include an option of a traditional, clinical healthcare provider 208 (such as a physician) providing the consumer 201 a prescription 209 (or a professional referral) for a DPP, which may include instructions for contacting the integrator 203. Some embodiments include an option of the integrator 203 providing a report 220 to the healthcare provider 208.

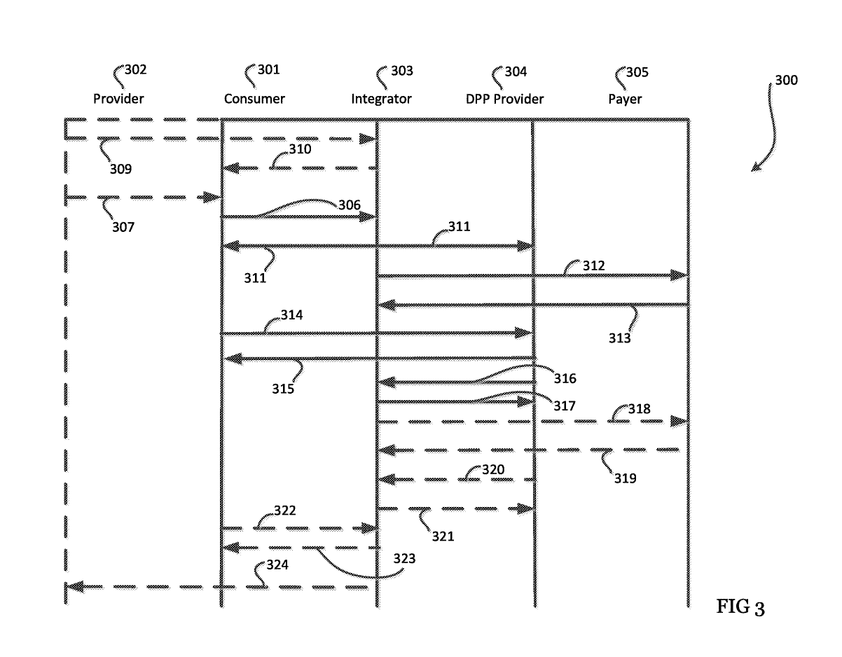

Various embodiments provide an augmented transition network system for optimizing placement of a consumer in a DPP, monitoring a status of consumer performance milestones while in the DPP, and sending payment to the DPP provider for completed milestones. Such embodiments include reporting the completed milestones to a payer, and receiving a payment from the payer triggered by the completed milestone. In an example, the augmented transition network system processes a series of events, as described below in connection with FIG. 3.

FIG. 3 is a process flow diagram 300 illustrating an exemplary use case involving a consumer 301, an integrator 303, a DPP provider 304, a payer 305, and optionally a healthcare provider 302. All of these parties have been described in detail in various portions of this application.

Some embodiments include an option of the healthcare provider 302, (for example, a doctor, a clinic, a hospital or a health care system) referring a consumer 301 to the integrator 303 (Step 309), whereupon the integrator 303 contacts the consumer 301 and invites the consumer 301 to log into the integrator's system (e.g., on-line portal) and find a DPP that is a best-fit for the consumer 301 (Step 310).

Some embodiments include an option of the healthcare provider 302 providing the consumer 301 with a prescription or other instruction to attend a DPP, which may include instructions for contacting the integrator 303 (Step 307).

In an embodiment the consumer 301 contacts the integrator 302 through a portal and provides various data points, such as, for example, the DPP program desired, personal information such as, name, address, zip code, associated payer 305 information, and the referral or prescription, which is used to set up an account in the integrator's system (Step 306). The integrator 303 walks the consumer 301 through a survey to create a health risk assessment and a matrix of personal preferences, which can include preferred modes of content delivery, location, time/days for sessions, group dynamics, virtual options, and the consumer's level of motivation to complete the DPP. The health risk assessment can be designed to provide Yes or No answers to whether or not the consumer 301 meets the criteria to be entered into the desired DPP. The matrix of personal preferences generates a personality profile, preferably including a location. If the consumer is eligible for one or more DPPs, the personality profile can be mapped against a plurality of ideal personality profiles associated with the DPP providers 304.

Using the results of the personality profile analysis and the consumer's location (e.g., home or work address), the integrator 303 determines which DPP provider 304 is the best-fit DPP provider 304 for the consumer 301. The integrator 303 enrolls the consumer 301 in the desired DPP with the best-fit DPP provider 304. The integrator 303 sends notice of the enrollment (which can include a DPP class schedule and any other information about the DPP, such as, dress code or dietary restrictions, or required monitoring systems) to both the consumer 301 and the DPP provider 304 (Step 311).

However, if the consumer 301 is already affiliated with a particular health plan from the payer 305, the integrator 303 may permit the payer or the consumer to designate a preferred DPP provider 304, as the best-fit DPP provider 304 for one or more of the DPPs for which the consumer is eligible.

Upon the enrollment of the consumer 301, the integrator 303 prepares and sends a claim to the payer 305 (Step 312). The integrator 303 receives an approval of the claim from the payer 305, which may include a partial payment claim (Step 313).

The consumer 301 participates in the DPP (Step 314). The DPP provider 304 provides the resources and delivers the content of the DPP to the consumer 301 (Step 315). As the consumer 301 progresses through the DPP, the DPP provider 304 updates the consumer's record and progress within a shared database maintained by the integrator 303 (Step 316).

In some embodiments, the integrator 303 may provide an interactive software tool for use by the DPP provider 304 to facilitate the integration process, for example, by allowing the DPP provider 304 to enter consumer data (e.g., attendance, body weight, and the like) directly into consumer's records maintained by, on behalf of, or at the direction of the integrator 303. In an embodiment, such an interactive software tool may include the Solera™ technology platform program available from Solera™ Health, Inc. located in Phoenix, Ariz.

Upon completion of the DPP or, alternatively, at various predetermined milestones, the integrator 303 makes a partial or full payment to the DPP provider 304 (Step 317).

In some embodiments, if multiple milestones are required for program completion, the system 30o can be setup to prepare and send one or more interim or supplemental claims to the payer 305 upon completion of each milestone (Step 318). The integrator 303 receives an approval of the interim or supplemental claim, which may include a partial payment (Step 319). The DPP provider 304 continues to update the consumer's record and progress within the shared database maintained by the integrator 303 (Step 320). Upon completion of the DPP or, alternatively, at the next predetermined milestone, the integrator 303 makes an additional partial or final payment to the DPP provider 304 (Step 321). The optional Steps 318 thru 321 can be repeated multiple times, as determined by the number of milestones that are in a particular DPP.

In some embodiments, the integrator 303 may send the consumer 301 a survey or otherwise solicit feedback at certain times during the DPP (Step 323). The consumer 301 completes the survey and the survey results are stored by the integrator 303. The survey can be directed to the quality and efficiency of the DPP and/or the DPP provider. Using machine learning capabilities of the integrator system, the results of a group of surveys can be analyzed to modify the ideal personality profile and/or other metrics for the DPP provider 304. In addition, the results of a group of surveys can be used to rank the DPP provider 304 among various DPP providers in a network.

In another embodiment, the consumer 301 can track data and milestones by accessing a dashboard provided by the integrator 303 (Step 322). The integrator 303 continually updates the data and populates the fields in the dashboard for viewing by the consumer (Step 323). In some embodiments, the integrator 303 can send one or more reports regarding the consumer's progress to the medical healthcare provider 302 (Step 324). The report can confirm successful completion of the DPP by the consumer 301 or, alternatively, can report the status if the DPP was not successfully completed. In this way the healthcare provider 302 can report aggregate quality metrics to Medicare/Medicaid agencies and the CDC, as well as chart and report the consumer's performance to the DPP.

In some embodiments, the integrator 303 reserves a portion of the payment from the payer 305, as compensation to the integrator for facilitating and managing the process. Alternatively, the payer 305 may pay a premium over the standard rate for the DPP in order to compensate the integrator 303 facilitating and managing the process. Typically, the premium paid by the payer 305 is less than the cost that the payer 305 would otherwise incur to facilitate and manage the process if the integrator 303 were not used, thus resulting in a net cost saving for the payer 305 in any event. In some embodiments, a set-up fee or a records fee may be included in the claim sent to the payer 305. This fee reimburses the integrator 303 for the costs of enrolling the consumer 301 in the DPP provided by the best-fit DPP provider and initiating an account for the consumer 301.

In some cases, a candidate consumer may be in need of more than one DPP. For example, candidate consumer may be in need programs for sleep disorder, obesity, and diabetes. The integrator system can be configured for the health risk assessment to recognize more than one disease and to identify corresponding DPPs. In one embodiment, the health risk assessment may be augmented by machine learning to analyze previous answers and determine if additional question strings need to be added to the assessment, to facilitate evaluating the consumer for other disease conditions. The integrator system can be configured to match a consumer to multiple DPPs and coordinate scheduling the multiple DPPs either simultaneously (all at once), sequentially, overlapping, or some together and others later in time.

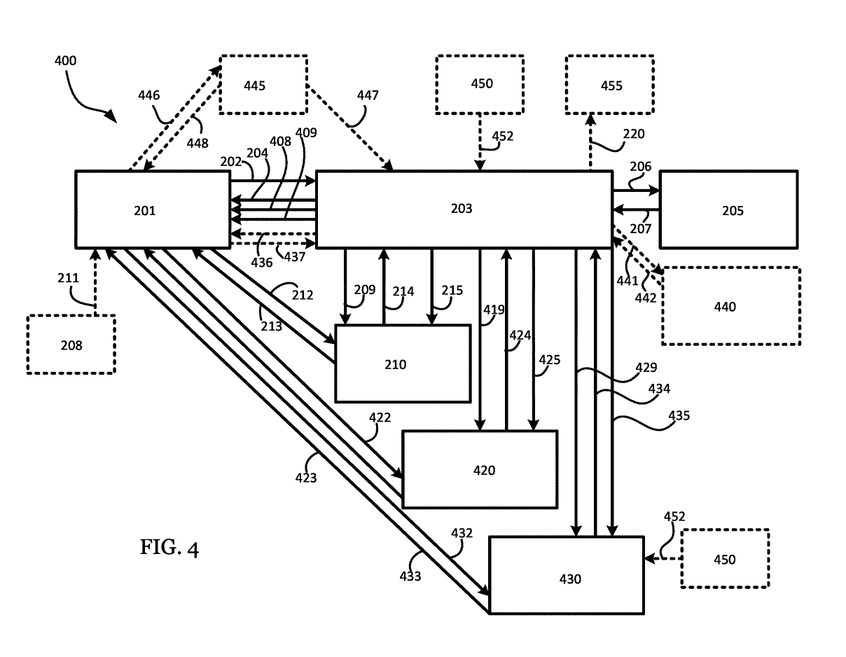

Turning now to FIG. 4, an exemplary system 400 for determining a plurality best-fit DPPs, each for a different disease, and a plurality of optimal DPP providers, for each disease, for a consumer 201 is shown.

As illustrated in FIG 4, the integrator 203 may be configured to facilitate a transaction or a series of inter-related transactions in a three-sided marketplace. In this regard, the consumer 201, a plurality of DPP providers 210, 420, 430 and the payer 205 (and an optional secondary payer 440) all interface with the integrator 203 at different events. Each of the transactions is distinguished by the individual DPP provider involved in the transaction. Stated in another way, only one DPP provider is typically involved with only one transaction. However, the consumer 201 can be involved in multiple transactions, with one or more DPP providers 210, 420, 430, and typically only one payer 205 for all of the transactions.

The transaction in the three-sided marketplace is driven by a series of events, where each event activates a change in system state (i.e. initiates an action, a task, or a process). The series of events and change of states can be computed by the integrator system employing a finite state machine or a modification thereof.

With continued reference to FIG. 4, the consumer 201 initiates the transaction by making contact with the integrator 203 for enrollment in a DPP. The consumer 201 inputs data 202 in response to a health risk assessment and to a personal preference survey. This data is analyzed by the integrator's computer system, for example, by algorithmically determining if the consumer 201 is eligible for one or more DPPs. The integrator's computer system can configure the health risk assessment to recognize more than one disease and identify corresponding DPPs. In one embodiment, the health risk assessment employs machine learning techniques to analyze previous answers and determine if additional question strings may be added to the assessment, to thereby evaluate the consumer for other disease conditions. If the consumer 201 is eligible for multiple DPPs, the integrator's computer system determines the best-fit DPP provider 210 for each DPP using analytics to compare the personal preferences of the consumer to the ideal preferences for each of the DPP providers. In addition, the respective locations of the consumer 201 and each of the DPP providers may also be considered. Based on this analysis of personal preferences and locations, a best-fit DPP provider 210 for each DPP is identified. The integrator 203 enrolls the consumer 201 in each of the best-fit DPPs with each best-fit DPP provider (first DPP provider 210, second DPP provider 420, and nth DPP provider 430). Notice of enrollment 204 is sent to the consumer 201. A second notice of enrollment is sent to each best-fit DPP provider (notice of enrollment 209 to first DPP provider 210, notice of enrollment 419 to second DPP provider 420, and notice of enrollment 429 to nth DPP provider 430).

Optionally, the health risk assessment can involve accessing the consumer's medical records, for example, if the consumer 201 sends permission 448 to medical record database 445 to authorize access 447 by integrator 203. The medical record database 445 can notify 446 consumers 201 that permission 448 for access 447 has been granted. Additionally, the medical record database 445 can notify 446 the consumer 201 each time the integrator 203 accesses 447 the medical record database 445. The integrator computer system may be configured to record one or more data points from the consumer's medical records (such as, for example: lab results, diagnosis, body fat, weight, blood components, and/or medications) and input the data points into the health risk assessment module.

Upon enrollment, the integrator 203 sends a claim 206 for each DPP to payer 205. The payer 205 sends an approval or payment 207 for each DPP to the integrator 203. In one embodiment, the consumer 201 has a secondary payer 440. Upon enrollment, the integrator 203 sends a claim 441 for an appropriate DPP to secondary payer 440. The secondary payer 205 sends an approval or payment 442 for the appropriate DDP to the integrator 203.

The consumer 201 participates 215 in the first DPP and the first DPP provider 210 delivers the DPP content 212 to the consumer 201. The DPP provider 210 reports 214 progress and other data to integrator 203. Once the integrator 203 identifies that the consumer 201 has satisfied a milestone or completed the DPP, the integrator 203 sends a payment 215 to the DPP provider 210, which ends the transaction or moves the transaction to achieving the next milestone.

Either simultaneously or at a different time, the consumer 201 participates 422 in the second DPP or the second DPP provider 420 delivers the second DPP content 423 to the consumer 201. The second DPP provider 420 reports 424 progress and other data to integrator 203. Once the integrator 203 identifies that the consumer 201 has accomplished a milestone or completed the second DPP, the integrator 203 sends a payment 425 to the second DPP provider 420, which ends the transaction or moves the transaction to the next milestone.

Finally, the consumer 201 participates 432 in the nth DPP and the nth DPP provider 430 delivers the nth DPP content 433 to the consumer 201. The nth DPP provider 430 reports 434 progress and other data to integrator 203. Once the integrator 203 identifies that the consumer 201 has satisfied a milestone or completed the nth DPP, the integrator 203 sends a payment 435 to the nth DPP provider 430, which ends the transaction or moves the transaction to the next milestone.

As discussed, a DPP provider reports data to the integrator relating to a consumer's participation in a DPP offered by the DPP provider. In one embodiment, one or more biometric devices 450 report 452 consumer data to nth DPP provider 430, which reads the consumer data and passes 434 the consumer data to the integrator 203. Alternatively, the biometric device 450 reports 452 the consumer data directly to the integrator 203. The biometric device 450 can be any device that can track, monitor, record and report progress of a task, condition, or biometric as the consumer 201 progresses through a DPP or a milestone thereof. Examples of a biometric 450 include, but are not limited to, a WI-FI enabled scale, a fit-bit, a pedometer, a sleep monitor, a blood pressure cuff, or any other sensor or transducer (wearable or standalone) capable of observing a physiological parameter.

Some embodiments include an option of a healthcare provider 208 providing the consumer 201 a prescription 209 (or a professional referral) for a DPP, which may include instructions for contacting the integrator 203. Some embodiments include an option of the integrator 203 providing a report 220 to a third-party 455 such as, for example, a healthcare provider, a court, a family member, a life coach, a probation officer, a twelve step sponsor, a doctor, an employer, or a government agency.

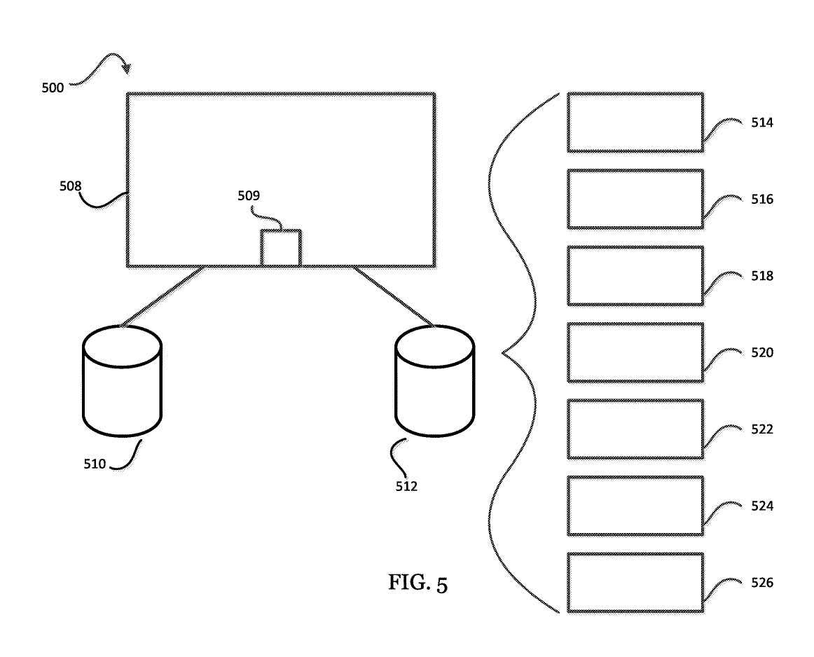

Referring now to FIG. 5, an integrator computer module 508 may be configured to perform any number of the various functions and tasks described herein. For example, a database system 500 includes an integrator computer module 508 having a processor or processing system 509. The integrator computer module 508 can be configured to maintain a first database 510 of DPP providers, and a second database 512 of candidate consumers; that is, the integrator builds and manages a relational database of health plan members. The integrator computer module 508 may be configured to recruit candidate consumers into the database 512 using at least the following sources (also referred to as entry vectors): employers 514, medical providers 516, health system 518, health plans 520, self-referral 522, network providers 524, and CBOs 526.

The integrator computer module 508 may be configured to import data sets from the foregoing vectors, stratify the data, and identify candidate consumers that fit a particular profile or otherwise have a need for one or more DPPs. In contrast, CBOs 526 are not typically equipped with the data security systems and protocols (e.g., HPPA compliant systems) or other processing infrastructure needed to securely manage large data sets.

With continued reference to FIG. 5, employers 514 may be brought into the system based on the fact that an employer's benefits package (e.g., an employee health insurance policy) has been revised to cover prevention programs.

Medical providers 516 may include doctors, groups of primary care physicians (PCP) whether geographically centralized or dispersed, pharmacists, and other health care professionals having a database of potential candidates for prevention programs.

Health systems 518 may include hospitals, hospital groups, primary and critical care facilities, nursing homes, rehabilitation centers, and outpatient facilities.

Health plans 520 may include health care insurance companies and their subsidiaries.

Self-referrals 522 may include consumers logging on to the integrator's web-site or other electronic portal, or responding to emails or other correspondence from the integrator.

Network providers 524 may include faith-based, community, non-profit, fraternal, social, athletic, and civil organizations such as Weight Watchers™, the YMCA™ and the YWCA™.

CBOs 526 may include county and community organizations, mental health services, and other social service agencies.

The foregoing sources may submit aggregate data to the integrator, whereupon the integrator analyses the data to determine eligibility and make program recommendations respecting qualifying consumers.

In an alternate embodiment, candidate consumers may be recruited into the system (i.e., into the integrator's database of candidate consumers) because a health plan initiates a call, email, or other communication to a candidate consumers. Those skilled in the art will appreciate that a health plan may be triggered to reach out to candidate consumers by patient costs exceeding a predetermined threshold, an emergency room visit, a claim for payment, an indication that the candidate consumer is not taking medications as prescribed, or another out of profile event or circumstance.

Moreover, in some circumstances it is advantageous to monitor the manner in which consumers enter the system. In particular, some protocols established by the Center for Disease Control (CDC) require that at least a certain percentage (e.g., 50%) of consumers enter the system by way of biometrics such as a blood test or other biometric, or a CPT code. In this regard, a Current Procedural Terminology (CPT) code set is a medical code set maintained by the American Medical Association through the CPT Editorial Panel. The remaining consumers may enter the system through soft protocols such as surveys, questionnaires, or other informal mechanisms. In this way the data set defined by the consumers may be deemed “valid” and, hence, credible, by the CDC.

The foregoing sources may submit aggregate patient data to the integrator, whereupon the integrator analyses the data to determine eligibility and make program recommendations for qualifying participants.

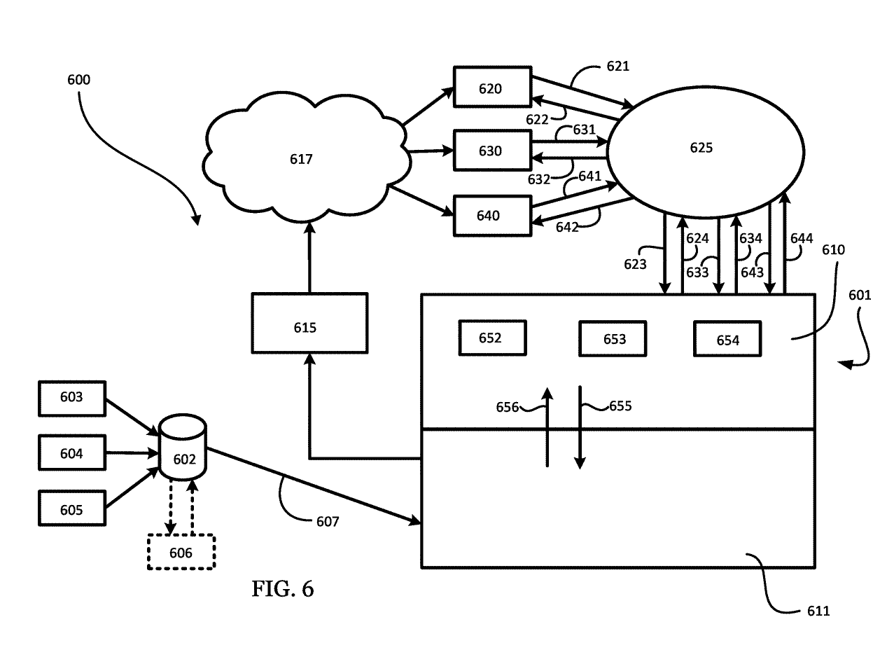

FIG. 6 illustrates a schematic block diagram of an exemplary integrator system 600, which includes an integrator application engine 610 configured to run on an integrator computer module 601.

An integrator computer module 601 comprises an integrator application engine 610 and a customer relationship management (“CRM”) system 611, which may be implemented as a software module. The integrator application engine 610 can receive 656 data from the CRM 611. Data from the integrator application engine 610 can send 655 data to populate fields in the CRM 611.

A database 602 can be configured to receive data from a plurality of sources including a first source 603, a second source 604, and an nth source 605. The first source 603, the second source 604, and the nth source 605 populate the database 602 with data from candidate consumers (participants), which can include name, contact information, email address, healthcare provider plan, and demographic data, such as, age, and ethnicity. In some embodiments, the first source 603, the second source 604, and the nth source 605 can be any of the sources described in FIG. 5. The database 602 operates within the framework of HIPAA and is protected with the appropriate firewalls and other safeguards to protect and limit access to the consumer data.

The data in the database 602 can be sent thru a scrubbing program 606, which can be configured to clean the data, fill in missing fields, and/or eliminate duplicates. The scrubbed data 607 is sent to the CRM 611 software module operating within the integrator computer module 601. CRM software modules are well known to those skilled in the art. For example, a Salesforce platform (Salesforce.com, San Francisco, Calif.) can be used as the CRM 611.

After the scrubbed data 607 is cataloged by the CRM 611, the CRM 611 sends a message including an attached weblink or other indicia of an integrator portal, along with at least a portion of the data relating to candidate consumers, to an email distribution module 615. Email distribution modules are well known to those skilled in the art. For example, a Marketo marketing automation platform (Marketo, Inc., San Mateo, Calif.) can be used as the email distribution module. The email distribution module 615 sends emails to a plurality of candidate consumers via the cloud 617 (e.g., the internet).

Upon receipt of the email, a candidate consumer is directed to a website 625 by clicking the weblink embedded in the email. A first consumer 620 opens the email and clicks the weblink, which puts the first consumer 620 in contact with the website 625. The first consumer 620 inputs data 621 based on a series of health risk assessment questions and a survey of personal preferences provided by the website 625. The received data 623 from the first consumer 620 is entered into the integrator application engine 610 for analysis. In some embodiments, the integrator application engine 610 can use machine learning to direct the health risk assessment questions based the received data 623. The integrator application engine 610 analyzes the received data 623 (which can include the answers to the health risk assessment questions and the survey of personal preferences, as well as physical characteristics such as, age, height, weight, sex, and ethnicity) and designs a personalized precision disease prevention program (“DPP”) 652 for the first consumer 620. The personalized precision DPP 652 is communicated 624 to the first consumer 620. The integrator application engine 610 can enroll the first consumer 620 into one or more personalized precision DPPs 652 and appropriate notices of enrollment 622 are sent to the first consumer 620 (and optionally to the DPP providers).

Similarly, a second consumer 630 interfaces with the website 625. The second consumer 630 inputs data 631 based on the health risk assessment questions and a survey of personal preferences. The received data 633 from the second consumer 630 is entered into the integrator application engine 610 for analysis. The integrator application engine 610 analyzes the received data 633 and designs a second personalized precision disease prevention program DPP 653 for the second consumer 620. The second personalized precision DPP 653 is communicated 634 to the second consumer 630. The integrator application engine 610 can enroll the second consumer 630 into the DPP program or programs associated with the second personalized precision DPP 653 and send appropriate notices of enrollment. Since the answers to the health risk assessment questions, the survey of personal preferences and physical characteristics will be different for the first consumer 620 and the second consumer 630, the personalized precision DPP 652 and the second personalized precision DPP 653 will likely be different and specific to each individual consumer.

As the number of consumers is theoretically infinitely scalable, an nth consumer 640 interfaces with the website 625. The nth consumer 640 inputs data 641 based on the health risk assessment personal preference survey. The received data 643 from the nth consumer 640 is entered into the integrator application engine 610 for analysis. The integrator application engine 610 analyzes the received data 643 and designs an nth personalized precision disease prevention program DPP 654 for the second consumer 620. The nth personalized precision DPP 654 is communicated 644 to the nth consumer 640. The integrator application engine 610 can enroll the nth consumer 640 into the nth personalized precision DPPs 654 and the notice of enrollment 642, along with instructions are sent to the nth consumer 640. Of course the nth personalized precision DPP 654, the personalized precision DPP 652, and the second personalized precision DPP 653 will inevitably be different and specific to each individual consumer.

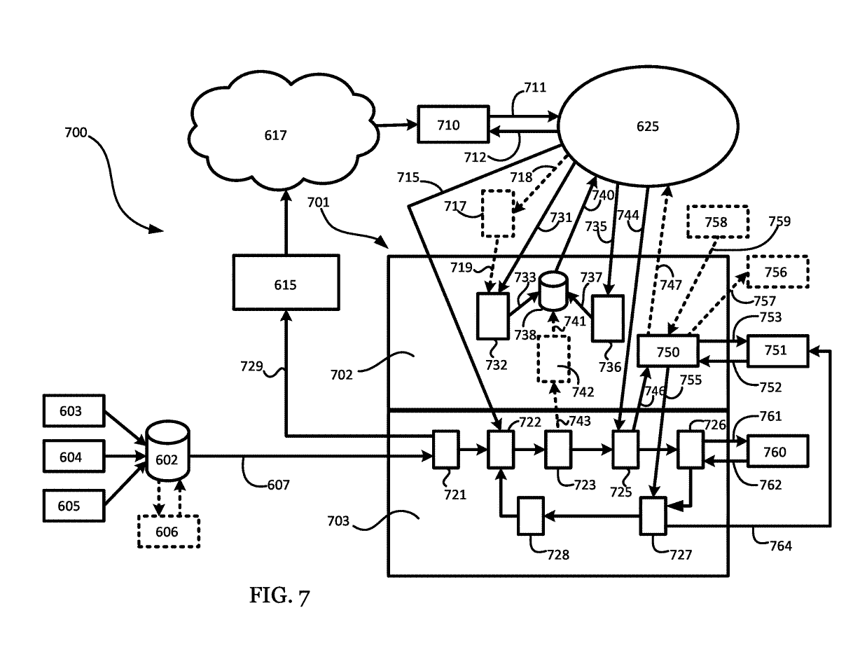

Turning now to FIG. 7, an exemplary integrator system 700 includes a more detailed implementation of an integrator application engine 702 and a CRM module 703.

More particularly, an integrator computer module 701 comprises an integrator application engine 702 and a CRM software module 703. The integrator application engine 702 can exchange data with the CRM. By way of non-limiting example, the integrator application engine 702 can send data to populate fields in the CRM 703.

In some embodiments, the CRM 703 comprises a listing module 721 configured to communicate with an account module 722; the account module 722 is configured to communicate with an opportunity module 723; the opportunity module 723 is configured to communicate with an enrollment module 725; the enrollment module 725 is configured to communicate with an invoicing module 726; the invoicing module 726 is configured to communicate with a fulfillment module 727; the fulfillment module 727 is configured to communicate with an asset module 728 which, in turn, is configured to communicate (directly or indirectly) with the account module 722.

In some embodiments, the integrator application engine 702 comprises a health risk assessment module 732, a personal profile module 736, an enterprise resource planning (“ERP”) module 750, a healthcare module 742, and a database 738 of DPP providers. Alternatively, the integrator application engine 702 may be configured to communicate with an external database 738 of DPP providers.

The scrubbed data 607 from the database 602 is sent to the CRM 702 software module operating within the integrator computer module 701. In particular, the scrubbed data 607 is sent to a list module 721 to be cataloged. The list module 721 creates cataloged data 729, which can include a message, a weblink, and at least a portion of the data relating to candidate consumers. The list module 721 sends the cataloged data 729 to an email distribution module 615. The email distribution module 615 sends emails using the cataloged data 729 to a plurality of candidate consumers via the cloud 617.

Upon receipt of the email, candidate consumers are directed to a website 625 by clicking the weblink embedded in the email. The consumer 710 enters contact information and/or healthcare plan data 715, which the system uses to create an account 722 in the CRM 703.

The consumer 710 inputs data 711 based on a series of health risk assessment questions, and the answers 731 are input into a health risk assessment module 732. In some embodiments, the consumer 710 can optionally contact 718 a health records database 717 and instruct the database 717 to directly download or otherwise input lab results and/or medical reports 719 into the risk assessment module 732. The risk assessment module 732 analyzes the answers 731 (and optionally medical records 719, which can include lab results, medical reports, and/or physical characteristics, such as, age, height, weight, sex, and ethnicity) and compares them to health criteria to determine if the consumer 710 is eligible for one or more DPPs. The resulting DPPs 733 are entered into a database 738.

The consumer 710 inputs data 711 into an interactive graphical user interface (GUI) hosted on the website 625 responsive to a survey of personal preferences, and the personal preference data 735 are input into a personal profile module 736. In addition, physical characteristics, such as, age, height, weight, sex, and ethnicity can be entered into the personal profile module 736. The personal profile module 736 analyzes the answers 735 and optionally the physical characteristics then sends the resulting personal profile 737 to the database 738.

Using the personal profile 737 and the consumer location, the integrator application engine 702 uses predictive analytics software algorithms to query the database 738 and determine which DPP provider or providers the resulting DPP 733, are the best-fit DPP providers for delivering the DPPs to the consumer 710.

The database 738 and appropriate software tools can use the personal profile 737 and resulting DPPs 733 to determine a best-fit DPP. Based on this analysis of the personal profile 737 and consumer location, a best-fit DPP provider or providers are identified.

In this regard, the integrator application engine 702 determines an ideal personality profile based on previous outcomes of the most successful participants for each DPP provider. Each DPP provider can have one or more vehicles for delivering a DPP. For example, a DPP provider can offer the DPP in a group session and can offer the DPP using a virtual interface.

However, if the consumer 710 is already affiliated with a particular health plan, the healthcare data is included in the opportunity module 723, which sends this information 743 to the healthcare module 742. The healthcare module 742 can designate one or more preferred DPP providers, as the best-fit DPP provider for one or more of the DPPs. The healthcare module 742 can send the list of preferred DPP providers 741 (also referred to as “white list”) to the database 738. The white list 741 limits the network of DPP providers available in the database 738, however, the integrator application engine 702 can also use predictive analytics to determine the best-fit DPP provider from among those listed in the white list 741.

Results 740 comprising one or more of the best-fit DPP the best-fit DPP providers are sent to the consumer 710 via the website 625. The website 625 enrolls 747 the consumer 710 in the best-fit DPP with the best-fit DPP provider 751. The website 625 sends the enrollment 747 to enrollment module 725. The website 625 sends notice of the enrollment 712 (which can include a DPP class schedule and any other information about the DPP, such as dress code policies, dietary restrictions, or required monitoring systems to the consumer 710. The enrollment module 725 sends notice of enrollment 746 to the ERP module 750, which forwards the enrollment to the DPP provider 751.

The ERP module 750 can be an interactive software tool for use by the DPP provider 751 to facilitate the integration process, for example, by allowing the DPP provider 751 to enter consumer data 752 (e.g., attendance, body weight, and the like) directly into consumer's records maintained in the integrator application engine 702. In one embodiment, a biometric device 758 reports consumer data 758 to the ERP module 75o. As described herein, the biometric 758 can be any device that can track and report progress of a task or condition, as the consumer 710 completes a DPP or a related milestone. In an embodiment, such an ERP module 750 may include the Solera™ technology platform program available from Solera™ Health, Inc. located in Phoenix, Ariz.

Upon enrollment, the enrollment module 725 sends notice to the invoicing module 726 to generate a claim 761 and send it to the payer 706. The payer 706 sends an approval or payment 762 to the invoicing module 726.

Once the ERP module 750 determines that the consumer 710 has satisfied a milestone or completed the DPP, the ERP module 750 notifies the fulfillment module 727, which in turn receives money or credit from invoicing module 726. The fulfillment module 727 sends a payment 764 to the DPP provider 753. The fulfillment module 727 sends notice to the asset module 728 to determine if services for the consumer 710 are complete; if not, the cycle continues.

In one embodiment, the ERP module 750 sends a report 757 to a third-party 756 which can be, for example, a healthcare provider, a court, an addiction recovery program, a family member, a doctor or a government agency.

In some embodiments, the integrator system uses series of algorithms to determine one or more “best-fit” programs for a candidate, thereby allowing the candidate to explore options based on his or her expressed preferences. Successful application of these insights can positively drive program engagement and influence health and wellness behaviors, and support ongoing retention, which significantly increases the probability of successful program completion.

In this context, “best-fit” implies meaningful engagement to ensure satisfaction of milestones, as well as successful DPP completion. Various examples of DPP programs include, inter alia, the following categories: i) lifestyle/prevention (pre-chronic); ii) chronic disease (e.g., congestive heart failure, arthritis, cavity prevention, falls prevention, diabetes, back pain, COPD, hypertension, cardiovascular disease); iii) behavioral health (e.g., addiction, domestic violence, anger management, depression, anxiety); and iv) pharmaceuticals, including compliance and dosage protocols.

In some embodiments, the integrator system can track meaningful engagement. As used herein, meaningful engagement refers to when the consumer participating in the DPP satisfies a milestone. Accordingly, meaningful engagement is typically an event which initiates an action such as sending a claim to the payer or sending payment to the DPP provider Meaningful engagement events can be tracked as monitored metrics and used to improve the system.

In some embodiments, the integrator system can implement segmentation as part of determining best fit DPPs and providers, which can involve analytic techniques to break down a heterogeneous population into smaller, homogeneous groups composed of individuals with similar needs, preferences, attitudes and behaviors. These segments are then analyzed with variables from a broader behavioral database (e.g., medical claims data). Unique, segment—specific variables may be isolated and extrapolated across a database population to flag each patient according to his or her segment. The integrator system can output the emergent segments, which can then be profiled against the “ideal” personal profile based on engagement and outcome data from each DPP provider.

The following table summarizes suitable exemplary segmentation criteria, some or all of which may be useful in the aforementioned segmentation process as well as defining personal profile for an individual candidate:

| Criteria | Example |
|---|---|
| Demographic | Age, gender, ethnicity, race, |
| and | education, income, and other physical |
| Socioeconomic | and situational characteristics. |
| | Zip code links to zip code specific |
| | social determinants of health and |
| | directory of local social services for |
| | cross - referrals. |
| Personal/ | Current co-morbid chronic health |
| Family Health | conditions, pregnancy status (if |
| Information | female), limitations to physical |
| and History | activity, Body Mass Index (height + |
| | weight), family history of disease |
| | (i.e. diabetes). |
| Behavioral/ | Current physical activity habits, |
| Attitudinal/ | current stress level, nutrition |
| Readiness | habits, tobacco use status. Readiness |
| | to change based on Prochaska's |
| | Transtheoretical Model. |
| Psychographic | Dimensions that identify motivations |
| Profile | and unarticulated needs. Attitudinal |
| | and behavioral variables based on |
| | the program/service/intervention. |
| | Includes values, beliefs, interests, |
| | principles, emotions, and personality. |
| | Includes needs and preferences for |
| | program services (flexibility, etc.). |
| Dieting | Number or dieting attempts, weight |
| History | loss goal, types of successful/ |
| | unsuccessful attempts. |
| Prescription | To determine, for example, use of |
| Use, History | Metformin for prediabetes or |
| and Compliance | prescription medication that may |
| | impact balance and increase likelihood |
| | of falls. |
| Health Care | For example, emergency room and |
| Utilization | hospitalizations or frequency of |
| and Behaviors | health care utilization and compliance |
| | with preventive exams. |
| Electronic | To determine program/service |
| Medical | intervention qualification. |
| Records or | |
| Claims Data | |

For example, qualifying criteria for a diabetes DPP may include: i) age over 18 and body mass index (BMI) over 24; and ii) age over 65 or a combination of being overweight and getting little exercise. In an embodiment, once eligibility is confirmed, the integrator system may present a drop down menu which allows the consumer to select his/her health plan. If not covered by a health plan, the consumer may elect to self-pay. The integrator system then starts collecting information (demographic, psychographic, medical, etc.) using branched logic. In one embodiment, a consumer can override the interactive data collection process and directly select a particular DPP, if desired.

In accordance with various embodiments, the integrator system programmatically determines an ideal consumer profile for the most successful participants for each DPP provider. For example, the integrator system identifies a first set of metrics common to the top graduates of a particular provider such as, for example, Jenny Craig™. The integrator system also identifies a second set of metrics common to the top graduates of Weight Watchers™, and so on. After gathering Patient Data (defined below) for a particular candidate, the integrator system employs machine learning and predictive analytics tool to recommend one or more “best fit” DPP programs (or multiple DPP programs) based on correlations between the candidate's personal profile and a plurality of ideal profiles.

Various embodiments of the present invention relate to integrator systems for: i) determining an “ideal” participant profile for each of a plurality of DPPs and/or DPP program providers based on the quality of consumer engagement and successful outcomes; ii) segmenting heterogeneous populations into homogeneous sub-groups to identify consumers who match the ideal profile; and iii) enrolling the identified consumer in the best fit DPP. Accommodating individual consumer needs and preferences in this way enables a more personalized approach to care, allowing health plans to engage with their members through prevention, thereby mitigating the higher long term costs of chronic disease treatment.

It is also in the payers' interest that the consumer successfully completes the DPP because it is generally cheaper to prevent chronic disease than to provide long-term treatment following full disease onset.

It should be noted that various embodiments described herein, while discussed in the context of Diabetes Prevention, are not so limited. Those skilled in the art will appreciate that the systems and methods described herein may contemplate any prevention or treatment program, as well as chronic disease management, telemedicine, medication and dosage adherence, social services, behavioral health, and the like.

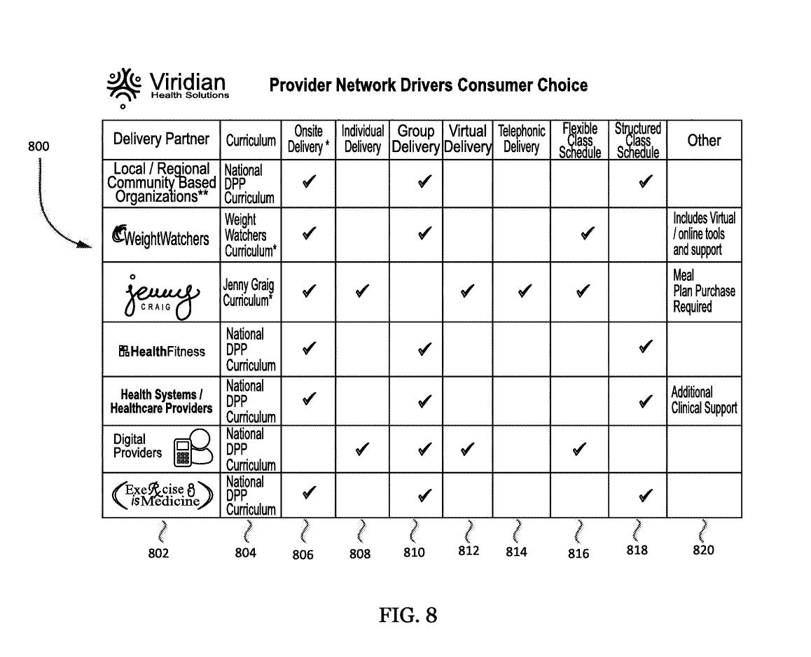

Referring now to FIGS. 8-13, various embodiments for determining a best fit DPP provider for a patient candidate will now be described in greater detail.

FIG. 8 is an exemplary spread sheet 800 of DPP providers (Delivery Partners) 802 within an integrator system, in accordance with various embodiments. More particularly, the spreadsheet 800 includes a number of columns identifies various delivery metrics associated with each DPP provider, including: type of curriculum 804; onsite delivery 806; individual delivery 808; group delivery 810; virtual delivery 812; telephonic delivery 814; flexible class schedule 816; structured class schedule 818; and other features 820 such as online tools/support, meal plans, and clinical support.

In conjunction with the foregoing, the following delivery metrics together comprise needs and preference variables (“NPVs”). In some embodiments, a NPV can be a type of intervention for the DPP. For example, the type of intervention can be one of or a combination of: online, mobile, text, telephonic, video-chat or in-person intervention. In this regard the integrator system choses the best-fit NPV based in a consumer's personal profile. In an exemplary configuration of this example, the integrator system can a NPV bank, which includes the following twelve types of intervention: i) one-on-one individual interventions or group-based program delivery; ii) group participation is optional or required; iii) groups are defined (the same people interact on a regular basis) and consistent, or groups are not defined (group membership varies from class to class); iv) content delivery and program participation is self-paced or follows a specific schedule; v) group participation is synchronous (e.g., weekly required webinar) or asynchronous (e.g., group interaction happens via chat or mobile discussion boards); vi) the intervention is led by a lay health educator or a clinician; vii) a member of the patient's clinical care team is incorporated into the program delivery vs the clinical care team receiving regular reporting from the program provider; viii) daily meal logging, taking pictures of food, volumetrics, or point systems; ix) frequency of health coach interaction (e.g., multiple times per day, daily, as needed, or weekly); x) patient's need for flexibility in day, time or location; xi) the method by which a standardized curriculum is delivered to the patient (e.g., video, quiz, printed materials); and xii) monitoring of weight, physical activity, medication and testing compliance or other biometrics via wearables and remote patient monitoring devices.

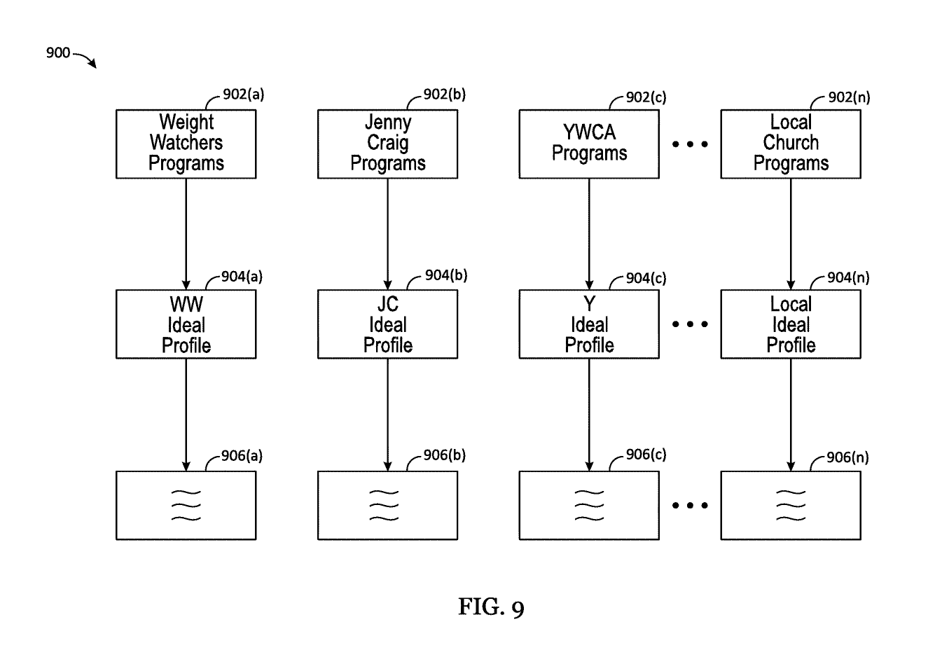

Referring now to FIG. 9, a schematic block diagram 900 illustrates a plurality of DPP providers 902(a)-902(n), each having an ideal profile 904(a)-904(n) associated therewith. In an embodiment, each ideal profile 904(a)-904(n) represents the common characteristics of participants who successfully completed that DPP's program, and may comprise a unique set of NPVs 906(a)-906(n). For example, a first set of NPVs 906(a) corresponding to DPP provider 902(a) may include in person, group based regular meetings having a specific schedule and led by a clinician; a second set of NPVs 906(b) corresponding to DPP provider 902(b) may include an on-line, mobile, self-paced program with daily meal logging and which uses a wearable device to monitor biometrics, and so on.

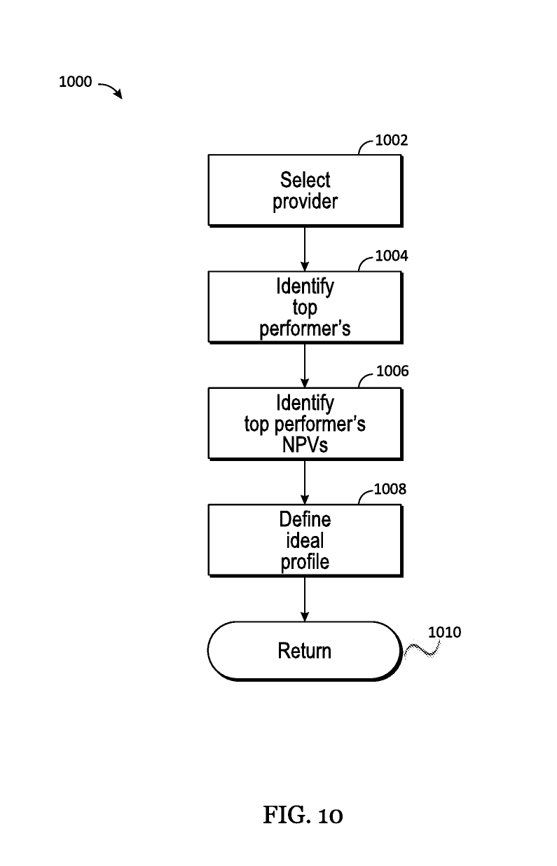

FIG. 10 is a flow diagram illustrating an exemplary process 1000 by which an integrator system can create ideal profiles for a plurality of DPP providers (which can be stored in a database). In particular, the process moo involves selecting (Task 1002) a DPP provider from a network of DPP providers, and identifying (Task 1004) top performers who successfully completed that DPP provider's program, based on engagement and outcome data. The process then identifies (Task 1006) common characteristics (e.g., NPVs) for the top performers. The DPP provider's ideal profile may then be defined (Task 1008) in terms of NPVs or other characteristics common to the DPP provider's top performers. The process moo then returns (Task 1010) and repeats the process to identify ideal profiles for additional DPP providers. Results from process 1000 can be stored on a database, which is accessible by integrator system.

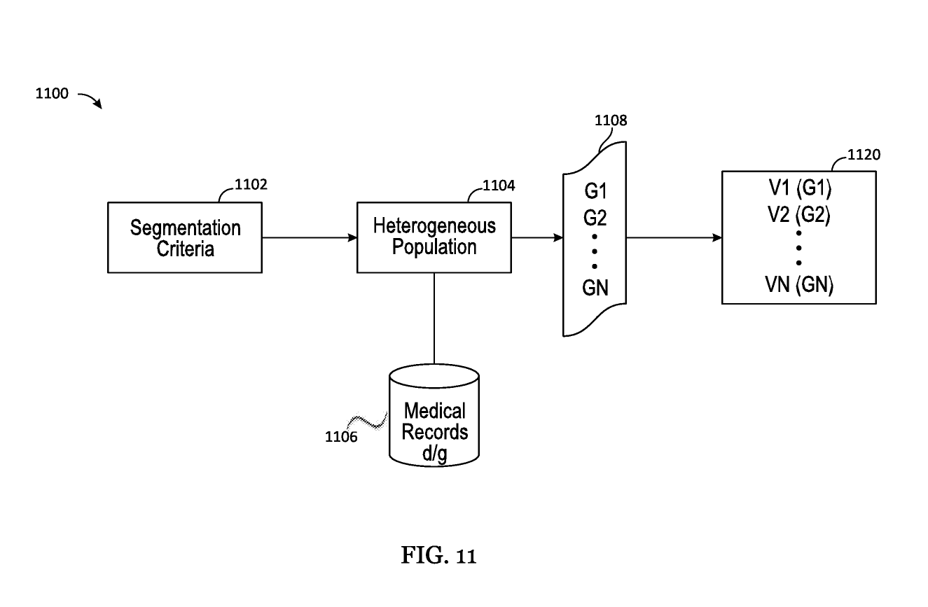

Returning now to the subject of segmentation, FIG. 11 is a schematic block diagram 1100 which illustrates a heterogeneous population broken down into smaller homogeneous sub-groups by an integrator system. More particularly, segmentation criteria 1102 may be used to filter data pertaining to heterogeneous population 1104, for example, population data residing in a medical or patient records database 1106 maintained by an integrator system, or by a health plan or a health care provider, which provide access to integrator system. Analytic techniques may be employed to break the population 1104 down into smaller, homogeneous groups 1108 (G1-GN), with each group G composed of individuals with similar personal preferences, such as, needs, preferences, attitudes and behaviors. The groups 1108 may be analyzed with variables from a broader behavioral database to which they are members (e.g. medical claims data) to isolate a set of segment-specific variables 1120 for each sub-group. That is, a first unique set of segment-specific variables V1(G1) may be determined for a first homogeneous sub-group G1, a second unique set of segment-specific variables V2(G2) may be determined for a second homogeneous sub-group G1, and so on.

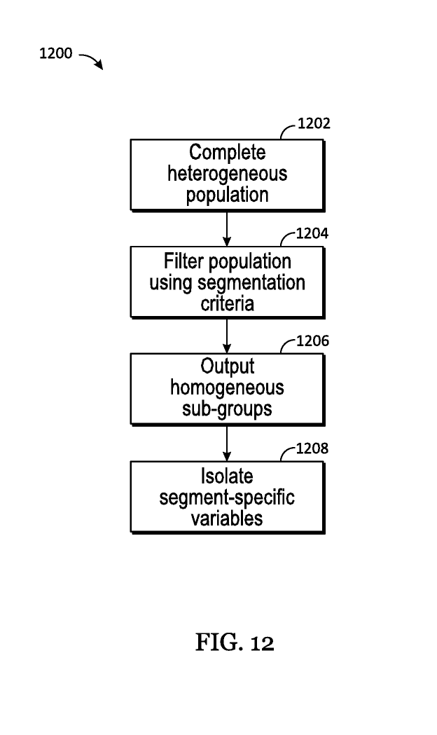

FIG. 12 is a flow diagram illustrating an exemplary process 1200 by which an integrator system segments a heterogeneous population into smaller homogeneous sub-groups characterized by segment-specific variables. In particular, the process 1200 involves the integrator system compiling (Task 1202) a database of heterogeneous population data, and using analytic techniques (Task 1204) to break down the heterogeneous population into smaller homogeneous sub-groups based on segmentation criteria, as discussed above. The process 1900 outputs (Task 1206) homogeneous sub-groups G1-GN of individuals, where the individuals within each sub-group share similar needs, attitude, preferences, and/or behaviors. Segment-specific variable may then be isolated (Task 1208) for each homogeneous sub-group.

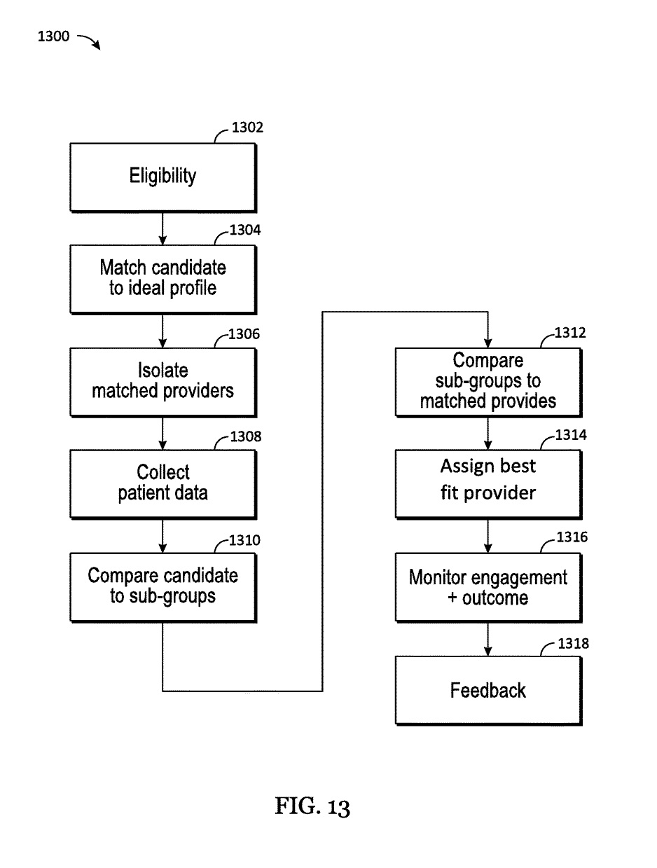

FIG. 13 is a flow diagram illustrating an exemplary process 1300 by which an integrator system selects a best fit provider for a candidate. More particularly, the process 1300 involves determining (Task 1302) whether a candidate is eligible for a DPP. The integrator system may be configured to ask the candidate questions, for example via branched logic, for use in comparing (Task 1304) the candidate with the ideal profiles discussed above in connection with FIGS. 9 and 10. In an embodiment, the integrator system may solicit NPV information from the candidate. In this way, the integrator system can compare the candidate NPVs to ideal profile NPVs to isolate (Task 1306) a set of matched ideal profiles for the candidate.

With continued reference to FIG. 13, the process 1300 further involves collecting (Task 1308) Patient Data from the candidate, and comparing (Task 1310) it to segment-specific variables from the homogeneous sub-groups discussed above in connection with FIGS. 11 and 12. Based on this comparison, the candidate may be assigned to one or more of the matching homogeneous sub-groups. The one or more matching sub-groups may then be compared (Task 1312) to the matched ideal profiles identified in Task 1306.

More particularly, the integrator system includes the profiles of the subgroup(s) assigned the ideal participant; the system eliminates unsuitable DPP providers based on a series of questions administered to the candidate relating to, for example, co-morbid conditions, BMI and dieting history, education, income, single-parent household, access to transportation, age, gender, race, ethnicity, language, the number and severity of chronic health conditions, the number of prescription medications and medication compliance, health behaviors including frequency of utilization of healthcare and preventive services, and other segmentation criteria.

Based on this comparison (Task 1312), the process 1300 assigns (Task 1314) the candidate to one or more best-fit DPP providers. The process 1300 employs machining learning, operating in the integrator system, to monitor (Task 1316) engagement and outcomes, and uses this information as feedback (Task 1318) to tune or refine any of the processes or parameters discussed herein such as, for example, creating subsequent ideal profiles.

The integrator system can be configured to employ a series of algorithms may be used to determine one or more “best-fit” programs for a candidate, thereby allowing the candidate to explore options based on his or her expressed preferences.

Various embodiments provide a method performed by a computer system for enrolling a consumer into a disease prevention program and paying a provider for the disease prevention program, the method includes the steps of: receiving contact from the consumer; performing a health risk assessment of the consumer; processing data from the health risk assessment against a set of minimum criteria for a plurality of disease prevention programs; identifying a disease prevention program based on a result from the processing data; surveying the consumer for personal preferences for the disease prevent program; creating a matrix of personal preferences for the consumer from surveying customer; comparing the matrix of personal preferences for the consumer against an ideal matrix of personal preferences for a plurality of providers, which are stored on a database in the computer system; determining a best fit provider for consumer based on the comparing the matrix; enrolling the consumer into the disease prevention plan with the best-fit provide; invoicing the consumer's health insurance plan for a claim to the disease prevention plan; receiving a first payment for the claim from the consumer's health insurance plan; tracking progress of the consumer in the disease prevention program; recognizing a milestone completed by consumer in the disease prevention program; and sending a second payment to the provider for in the disease prevention program.

The method can include the step of providing the consumer a notice of enrollment in the disease prevention program with the best-fit provider.

The notice of enrollment can also include at least one of a set of instructions for the plan, a class schedule for the plan, clothing requirements for the plan, and dietary restrictions for the plan.

The method can include the step of providing the provider a notice of enrollment of the consumer in the disease prevention program.

The method can include the step of determining if the consumer is eligible for the disease prevention based on a result from the from the health risk assessment.

The method can also include the step of terminating contact with the consumer if the consumer is not eligible for the disease prevention program.

The method can include the steps of: comparing the result from the health risk assessment to ideal results for a plurality of prevention programs; and determining a best-fit disease prevention program for the consumer based on the comparing the result.

The method can include the steps of: identifying a location of a residence of the consumer; comparing the location consumer against a location of a plurality of providers, which are stored on a database in the computer system; and determining a best fit provider for consumer based on the comparing the matrix and the comparing the location.

The method can include the step of retaining a portion of the payment for the claim from the consumer's health insurance plan.

The payment for the claim from the consumer's health insurance plan includes a premium above the payment to the provider for in the disease prevention program.

The payment for the claim from the consumer's health insurance plan includes a records fee for an account for the consumer.

Various embodiments provide a computer system for coordinating integrating transactions in a three-sided marketplace consisting of a consumer, a disease prevention program provider, and a health insurance plan provider, the computer system can include: an integrator application engine; and a customer relationship management module; wherein the customer relationship management module comprises a listing module, an account module, an opportunity module, an enrollment module, an invoicing module, a fulfillment module, and an asset module; wherein the listing module a listing module is configured to communicate with an account module; the account module is configured to communicate with an opportunity module; the opportunity module is configured to communicate with an enrollment module; the enrollment module is configured to communicate with an invoicing module; the invoicing module is configured to communicate with a fulfillment module; the fulfillment module is configured to communicate with an asset module which, in turn, is configured to communicate (directly or indirectly) with the account module; wherein the integrator application engine comprises a health risk assessment module, a personal profile module, and an enterprise resource planning module; wherein the enrollment module is in communication with the enterprise resource planning module; and wherein the enterprise resource planning module is in communication with the fulfillment module.

The computer system can be further configured for: the consumer to be in communication with the account module, the health risk assessment module, the personal profile module, and the enrollment module; the a disease prevention program provider to be in communication with the enterprise resource planning module, and the fulfillment module; and the health insurance plan provider to be in communication with the invoicing module.

The computer system can be further configured to: send a payment to the facilitator by the health insurance plan via to the invoicing module; and send a payment for the program to the disease prevention provider by the facilitator via the fulfillment module.

The computer system can be further configured to: receive consumer progress data from the disease prevention provider via the enterprise resource planning module; and initiate the payment for the program upon the fulfillment module receives a notice of a completed milestone from the enterprise resource planning module.

The health risk assessment module can be configured to: perform a health risk assessment of the consumer; process data from the health risk assessment against a set of minimum criteria for a plurality of disease prevention programs; and identify a disease prevention program based on a result from the processing data.

The personal profile module can be configured to: provide a survey of personal preferences; collect answers to the survey from the consumer; analyze the answers to the survey; and create a personal profile for the consumer.

The integrator application engine can be configured to: identify a disease prevention program for the consumer; access a database of a plurality of disease prevention program providers offering the disease prevention program; comparing using predictive analytics the personal profile for the consumer to ideal personal preferences for the plurality of disease prevention program providers, which are stored on the database; and determining a best-fit disease prevention program provider for consumer based on the comparing of the personal profile.

Various embodiments provide a computer code stored in a non-transient medium for performing, when executed by a computer processor, the steps of: receiving contact by the computer system from the consumer; performing a health risk assessment of the consumer; processing data from the health risk assessment against a set of minimum criteria for a plurality of disease prevention programs; identifying a disease prevention program based on a result from the processing data; surveying consumer for personal preferences for the disease prevent program; creating a matrix of personal preferences for the consumer from surveying customer; comparing the matrix of personal preferences for the consumer against an ideal matrix of personal preferences for a plurality of providers, which are stored on a database in the computer system; determining a best fit provider for the consumer based on the comparing the matrix; enrolling the consumer into the disease prevention plan with the best-fit provider; invoicing the consumer's health insurance plan for a claim to the disease prevention plan; receiving a first payment for the claim from the consumer's health insurance plan; tracking progress of the consumer in the disease prevention program; recognizing a milestone completed by the consumer in the disease prevention program; and sending a at least a portion of a final payment to the provider upon recognizing the completed milestone.

As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated.

As used herein, the phrase “at least one of A, B, and C” can be construed to mean a logical (A or B or C), using a non-exclusive logical “or,” however, can be contrasted to mean (A, B, and C), in addition, can be construed to mean (A and B) or (A and C) or (B and C). As used herein, the phrase “A, B and/or C” should be construed to mean (A, B, and C) or alternatively (A or B or C), using a non-exclusive logical “or.”

It should be understood that steps within a method may be executed in different order without altering the principles of the present disclosure. For example, various embodiments may be described herein in terms of various functional components and processing steps. It should be appreciated that such components and steps may be realized by any number of hardware components configured to perform the specified functions.

While the foregoing detailed description will provide those skilled in the art with a convenient road map for implementing various embodiments of the invention, it should be appreciated that the particular embodiments described above are only examples, and are not intended to limit the scope, applicability, or configuration of the invention in any way. To the contrary, various changes may be made in the function and arrangement of elements described without departing from the scope of the invention.

What is claimed:

1. A method performed by a computer system for enrolling a consumer into a disease prevention program and paying a provider for the disease prevention program, the method comprising:
receiving contact from the consumer;
performing a health risk assessment of the consumer;
processing data from the health risk assessment against a set of minimum criteria for a plurality of disease prevention programs;
identifying a disease prevention program based on a result from the processing data;
surveying the consumer for personal preferences for the disease prevent program;
creating a matrix of personal preferences for the consumer from surveying the consumer;
comparing the matrix of personal preferences for the consumer against an ideal matrix of personal preferences for a plurality of providers, which are stored on a database in the computer system;
determining a best fit provider for the consumer based on comparing the matrix;
enrolling the consumer into the disease prevention plan with the best-fit provider;
invoicing the consumer's health insurance plan for a claim to the disease prevention plan;
receiving a first payment for the claim from the consumer's health insurance plan;
tracking progress of the consumer in the disease prevention program;
recognizing a milestone completed by the consumer in the disease prevention program; and
sending at least a portion of the first payment to the provider upon recognizing the completed milestone.

2. The method according to claim 1, further comprising the step of providing the consumer a notice of enrollment in the disease prevention program with the best-fit provider.

3. The method according to claim 2, wherein notice of enrollment comprises at least one of a set of instructions for the plan, a class schedule for the plan, clothing requirements for the plan, and dietary restrictions for the plan.

4. The method according to claim 1, further comprising the step of providing the provider a notice of enrollment of the consumer in the disease prevention program.

5. The method according to claim 1, further comprising the step of determining if the consumer is eligible for the disease prevention based on a result from the from the health risk assessment.

6. The method according to claim 5, further comprising the step of terminating contact with the consumer if the consumer is not eligible for the disease prevention program.

7. The method according to claim 1, further comprising the steps of
comparing the result from the health risk assessment to ideal results for a plurality of prevention programs; and
determining a best-fit disease prevention program for the consumer based on the comparing the result.

8. The method according to claim 1, further comprising the steps of
identifying a location of a residence of the consumer;
comparing the location consumer against a location of a plurality of providers, which are stored on a database in the computer system; and
determining a best fit provider for consumer based on the comparing the matrix and the comparing the location.

9. The method according to claim 1, further comprising the step of retaining a portion of the first payment for the claim from the consumer's health insurance plan.

10. The method according to claim 9, wherein the payment for the claim from the consumer's health insurance plan includes a premium above the first payment to the provider for in the disease prevention program.

11. The method according to claim 9, wherein the first payment for the claim from the consumer's health insurance plan includes a records fee for an account for the consumer.

12. The method according to claim 1, wherein the provider is a non-medical entity.

13. A computer system for coordinating transactions in a three-sided marketplace consisting of a consumer, a disease prevention program provider, and a health insurance plan provider, the computer system comprising:
an integrator application engine; and
a customer relationship management module;
wherein the customer relationship management module comprises a listing module, an account module, an opportunity module, an enrollment module, an invoicing module, a fulfillment module, and an asset module;
wherein the listing module is configured to communicate with an account module;
wherein the account module is configured to communicate with an opportunity module;
wherein the opportunity module is configured to communicate with an enrollment module;
wherein the enrollment module is configured to communicate with an invoicing module;
wherein the invoicing module is configured to communicate with a fulfillment module;
wherein the fulfillment module is configured to communicate with an asset module;
wherein the asset module is configured to communicate with the account module;
wherein the integrator application engine comprises a health risk assessment module, a personal profile module, and an enterprise resource planning module;
wherein the enrollment module is in communication with the enterprise resource planning module; and
wherein the enterprise resource planning module is in communication with the fulfillment module.

14. The computer system according to claim 13, wherein the computer system is further configured for:
the consumer to be in communication with the account module, the health risk assessment module, the personal profile module, and the enrollment module;
the a disease prevention program provider to be in communication with the enterprise resource planning module, and the fulfillment module; and
the health insurance plan provider to be in communication with the invoicing module.

15. The computer system according to claim 14, wherein the computer system is further configured to:
send a payment to the facilitator by the health insurance plan via to the invoicing module; and
send a payment for the program to the disease prevention provider by the facilitator via the fulfillment module.

16. The computer system according to claim 15, wherein the computer system is further configured to:
receive consumer progress data from the disease prevention provider via the enterprise resource planning module; and
initiate the payment for the program upon the fulfillment module receiving a notice of a completed milestone from the enterprise resource planning module.

17. The computer system according to claim 13, wherein the health risk assessment module is configured to:
perform a health risk assessment of the consumer;
process data from the health risk assessment against a set of minimum criteria for a plurality of disease prevention programs; and
identify a disease prevention program based on a result from the processing data.

18. The computer system according to claim 13, wherein the personal profile module is configured to:
provide a survey of personal preferences,
collect answers to the survey from the consumer;
analyze the answers to the survey; and
create a personal profile for the consumer.

19. The computer system according to claim 18, wherein the integrator application engine is configured to:
identify a disease prevention program for the consumer;
access a database of a plurality of disease prevention program providers offering the disease prevention program;
compare using predictive analytics the personal profile for the consumer to ideal personal preferences for the plurality of disease prevention program providers, which are stored on the database; and
determine a best-fit disease prevention program provider for consumer based on the comparing of the personal profile.

20. Computer code stored in a non-transient medium for performing, when executed by a computer processor, the steps of:
receiving contact from the consumer;
performing a health risk assessment of the consumer;
processing data from the health risk assessment against a set of minimum criteria for a plurality of disease prevention programs;
identifying a disease prevention program based on a result from the processing data;
surveying the consumer for personal preferences for the disease prevent program;
creating a matrix of personal preferences for the consumer from the surveying consumer;
comparing the matrix of personal preferences for the consumer against an ideal matrix of personal preferences for a plurality of providers, which are stored on a database;
determining a best fit provider for consumer based on comparing the matrix;
enrolling the consumer into the disease prevention plan with the best-fit provider;
invoicing the consumer's health insurance plan for a claim to the disease prevention plan;
receiving a first payment for the claim from the consumer's health insurance plan;
tracking progress of the consumer in the disease prevention program;
recognizing a milestone completed by the consumer in the disease prevention program; and
sending at least a portion of the first payment to the provider upon recognizing the completed milestone.
